(19) United States
(12) Patent Application Publication
(10) Pub. No.: US 2026/0335279 A1
(43) Pub. Date: Oct. 29, 2026

(54) METHODS OF TREATING ISCHEMIA-REPERFUSION INJURIES

(71) Applicant: University of Cincinnati, Cincinnati, OH (US)

(72) Inventor: Konstantinos Drosatos, Cincinnati, OH (US)

(73) Assignee: University of Cincinnati, Cincinnati, OH (US)

(21) Appl. No.: 19/108,992

(22) PCT Filed: Sep. 7, 2023
(86) PCT No.: PCT/US2023/073630

§ 371 Date: Mar. 5, 2025

Related U.S. Application Data
(60) Provisional application No. 63/404,459, filed on Sep. 7, 2022.

(51) Int. Cl. A61K 31/382; A61K 31/12; A61K 31/496; A61K 31/517; A61P 9/10

(52) U.S. Cl. CPC A61K 31/382; A61K 31/12; A61K 31/496; A61K 31/517; A61P 9/10

(57) ABSTRACT

Methods of treating a patient in need of reperfusion therapy following an ischemic event are provided herein. The method generally involves administering to the patient one or more primary therapeutic agents in combination with onset of reperfusion therapy, wherein the one or more primary therapeutic agents are selected from the group consisting of Krüppel-like factor 5 (KLF5) inhibitors, NADPH oxidase (NOX) inhibitors, and glucose transport (GLUT) inhibitors.

### CROSS REFERENCE TO RELATED APPLICATION

This application claims priority to U.S. Provisional Application Ser. No. 63/404,459, filed Sep. 7, 2022, the entire contents of which are incorporated herein by reference.

### SEQUENCE LISTING

A computer-readable form (CRF) sequence listing having file name Sequence_Listing_10738_1065.xml (16,608 Bytes), created on Sep. 5, 2023, is incorporated herein by reference. The nucleic acid sequences listed in the accompanying sequence listing are shown using standard abbreviations as defined in 37 C.F.R. § 1.822.

### TECHNICAL FIELD

The present disclosure relates generally to the field of medical therapeutics, specifically compositions and methods for protecting cardiac tissue from reperfusion injury.

### BACKGROUND

Acute myocardial infarction (AMI) is the leading cause of disability and death worldwide. In patients with AMI, the treatment of choice for reducing AMI injury and limiting infarct size (IS) is timely and effective myocardial reperfusion, which can be achieved using either thrombolytic therapy or primary percutaneous coronary intervention (PCI). Paradoxically, restoration of coronary blood flow itself can further provoke cardiomyocyte death, confining the beneficial effect of optimal reperfusion. This phenomenon, termed myocardial reperfusion injury, for which there is still no effective treatment, accounts for up to 50% of the final myocardial infarct.

Ischemia-reperfusion injuries are tissue damage resulting from blood supply being restored to a tissue or organ after a period of ischemia. Generally, the absence of oxygen and nutrients from blood during the ischemic period leads to an increase in waste products and disruption of normal cellular processes. Restoration of circulation results in inflammation and oxidative damage through the induction of oxidative stress. Myocardial ischemia-reperfusion injury can result in reversible functional myocardial deterioration (i.e., stunning) and/or irreversible tissue damage (i.e., infarction and fibrosis).

Different biochemical and metabolic changes have been accused of mediating myocardial reperfusion injury, including the generation of reactive oxygen species (ROS), the Ca²⁺ overload, the abrupt restoration of physiological pH, and the inflammatory response. These changes promote cardiomyocyte death through multiple pathways, which among others include apoptosis, necrosis, pyroptosis, autophagy, and ferroptosis. However, their exact contribution, as well as the time point at which each specific cell death mode occurs during reperfusion, still remain elusive.

In molecular genetics, the Krüppel-like factors (KLFs) coupled with the Sp transcription factors, forming the Sp/KLF, are a family of 26 transcription factors that contain three C2H2-zinc finger motifs that regulate gene expression. KLF5, also known as intestinal enriched Krüppel-like factor (IKLF) or basic transcription element binding protein 2 (Bteb2), has been shown to regulate cardiac fatty acid oxidation via direct activation of PPARα (peroxisome proliferator-activated receptor α). Previous studies have shown that KLF5 is induced during ischemic events and increases ceramide production. However, mice with cardiomyocyte-specific deletion of KLF5 have lower cardiac fatty acid oxidation rates and show an increased risk of heart failure. However, prior to the instant disclosure, the relationship between KLF5 and ischemia-reperfusion injuries has not been appreciated.

Accordingly, a need exists to identify the pathophysiological mechanisms by which ischemia-reperfusion injuries occur and to develop treatments that protect cardiac tissue from cellular damage during reperfusion therapy, and reduce mortality and morbidity associated with oxidative injury.

### SUMMARY

The instant disclosure elucidates the pathological mechanism of KLF5 and its target genes in cardiotoxicity and ischemia-reperfusion injuries. Further, the instant disclosure elucidates the role of KLF5 inhibition and/or the target gene inhibition in cardioprotection, more specifically, in reducing infarct size and rescuing cardiac tissue. Accordingly, provided herein are methods for the treatment of ischemia-reperfusion injuries comprising the administration of KLF5 inhibitors and/or inhibitors of the downstream elements, namely NOX4 and GLUT.

Example embodiments disclosed herein are directed to methods of treating a patient in need of reperfusion therapy following an ischemic event, the method comprising administering to the patient one or more primary therapeutic agents in combination with onset of reperfusion therapy, wherein the one or more primary therapeutic agents are selected from the group consisting of Krüppel-like factor 5 (KLF5) inhibitors, NADPH oxidase (NOX) inhibitors, and glucose transport (GLUT) inhibitors. Optionally, the patient has suffered a myocardial infarction. In some embodiments, the one or more primary therapeutic agents is a KLF5 inhibitor, such as ML264, SR18662, SR15006, and CID 5951923, optionally ML264. In some embodiments, the one or more primary therapeutic agents is a NOX inhibitor or a NOX4 inhibitor, optionally GLX-351322, apocynin, or GKT-137831. In some embodiments, the one or more primary therapeutic agents is a GLUT inhibitor, such as KL-11743, cyctochalasin B, or cytochalasin E, optionally KL-11743. The method optionally involves administering a plurality of primary therapeutic agents. In some embodiments, the one or more primary therapeutic agents are administered systemically via an enteral or parenteral route. The one or more primary therapeutic agents is optionally administered as a single dose or in multiple doses. In some embodiments, a first dose of the multiple doses is administered before the onset of reperfusion therapy, while a second dose of the multiple doses is administered after the onset of reperfusion therapy.

In another embodiment, the present disclosure is directed to a method of treating a patient experiencing a myocardial ischemic event, the method comprising administering to the subject an effective amount of one or more primary therapeutic agents selected from the group consisting of ML264, apocynin, KL-11743, and GLX-351322, prior to administering reperfusion therapy to the patient. Optionally, second dose of the one or more primary therapeutic agents may be administered after onset of reperfusion therapy.

These and other features, aspects, and advantages will become better understood with reference to the following description and the appended claims.

Additional features and advantages of the embodiments described herein will be set forth in the detailed description that follows, and in part will be readily apparent to those skilled in the art from that description or recognized by practicing the embodiments described herein, including the detailed description that follows, the claims, as well as the appended drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1D demonstrate that cardiomyocyte KLF5 is transiently induced early in cardiac reperfusion upon myocardial ischemia-reperfusion injury. FIG. 1A graphically illustrates the mRNA expression levels of KLF5, Ppara, and NOX4 at various durations of reperfusion therapy. FIG. 1B graphically illustrates the total protein expression level of KLF5 at the same time points of FIG. 1A. Investigation of the KLF5 mRNA levels on the RNA isolated from each cell population, revealed a significant increase in KLF5 mRNA levels only in cardiomyocytes derived from the I/R mice, as demonstrated by FIG. 1C. This result was further confirmed at a protein level, using protein lysate isolated from pure cardiomyocytes (FIG. 1D).

FIGS. 2A-2J demonstrate that cardiomyocyte-specific KLF5 genetic deletion reduces infarct size and protects the heart upon I/R injury. The αMHC-KLF5^{-/-} were subjected to 30 minutes of myocardial ischemia and 24 hours of reperfusion therapy, for the concurrent evaluation of cardiac function (FIG. 2A) and infarct size, compared with WT (αMHC-Cre+/−). Cardiac 2D echocardiography analysis showed a higher ejection fraction in the αMHC-KLF5^{-/-} mice subjected to reperfusion therapy compared to the WT mice undergoing reperfusion (FIG. 2B). Speckle tracking echocardiography revealed a significant increase in lateral wall radial strain (FIG. 2C), lateral wall radial strain rate (FIG. 2D), lateral wall circumferential strain (FIG. 2E) and lateral wall circumferential strain rate (FIG. 2F) and global circumferential strain (FIG. 2G) in the αMHC-KLF5^{-/-} mice subjected to reperfusion therapy compared to the WT mice undergoing reperfusion. Consistently, infarct size evaluation upon Evan's Blue/TTC staining (FIG. 2H) revealed a significant reduction of infarct size in the αMHC-KLF5^{-/-} group, compared to the WT groups (FIG. 2I, FIG. 2J).

FIGS. 3A-3K demonstrate that pharmacological inhibition of KLF5 preserves cardiac function and reduces infarct size upon ischemia-reperfusion injury. KLF5 suppression was confirmed in the cardiac tissue at 30 minutes of ischemia by the mRNA expression level (FIG. 3A) and the protein expression (FIG. 3B). A significant preservation of ejection fraction was observed in I/R mice treated with ML264, compared to those treated with the vehicle (FIG. 3C). Speckle tracking echocardiography revealed a significant increase in lateral wall radial strain (FIG. 3D), lateral wall radial strain rate (FIG. 3E), lateral wall circumferential strain (FIG. 3F) and lateral wall circumferential strain rate (FIG. 3G) and global circumferential strain (FIG. 3H) in the ML264-treated mice, in comparison to mice treated with the vehicle. Consistently, infarct size evaluation upon Evan's Blue/TTC staining (FIG. 3I) revealed a significant reduction of infarct size in the ML264-treated mice, in comparison to mice treated with the vehicle (FIG. 3J, FIG. 3K).

FIGS. 4A-4D demonstrate that acute pharmacological as well as chronic KLF5 inhibition attenuates cardiac dysfunction and fibrosis in a chronic model of I/R injury. 2D echocardiography revealed a significant preservation in ejection fraction in the ML264 and the αMHC-KLF5-groups at 4 weeks post-I/R, in comparison to the IR group treated with the vehicle (FIG. 4A). Masson's Trichrome staining was performed to the heart sections for the investigation of cardiac fibrosis and revealed a significant reduction of fibrotic scar tissue formation both in the ML264 and the αMHC-KLF5^{-/-} groups (FIG. 4B-4C). mRNA levels of Col1a1 and Col3a1 were also decreased in these two groups, in comparison to the IR group treated with the vehicle. Importantly, KLF5 mRNA levels were unchanged among the four groups at 4 weeks post-ischemia-reperfusion, indicating that KLF5 inhibition at early reperfusion is sufficient to ameliorate the cardiotoxic effect of KLF5 in myocardial ischemia-reperfusion injury (FIG. 4D).

FIGS. 5A-5R demonstrate that KLF5 promotes oxidative stress and leads to apoptosis through NOX4-mediated mechanisms. DHE staining (FIG. 5A) revealed a significant reduction in reactive oxygen species formation in the ML264-treated group, in comparison to the ischemia-reperfusion group treated with the vehicle (FIG. 5B). Further NOX4 mRNA (FIG. 5C) and protein (FIG. 5D (representative western blot) and FIG. 5E) levels were suppressed upon ML264 treatment. Investigation of the apoptotic pathway revealed a suppression of the BAX apoptotic proteins (FIG. 5F) and an upregulation of the BCL-XL anti-apoptotic proteins (FIG. 5G) in the ML264 treated group, indicating that KLF5 inhibition at early reperfusion ameliorates ischemia-induced apoptosis (FIG. 5H). Given the fact that inflammation and diapedesis of immune cells are important hallmarks of the post-ischemia-reperfusion response, the mRNA levels of IL-1α (FIG. 5I), IL-1β (FIG. 5J), IL-6 (FIG. 5K), IL-10 (FIG. 5L), and TNFα (FIG. 5M) in the ischemic myocardium upon ML264 treatment were evaluated at 2 hours of reperfusion (FIGS. 5I-5M) and 24 hours of reperfusion (FIGS. 5N-5Q: IL-1α (FIG. 5N), IL-1β (FIG. 5O), IL-6 (FIG. 5P), IL-10 (FIG. 5Q), and TNFα (FIG. 5R)). Significant suppression of IL-1β, IL-6, and TNFα levels in the ML264 group was observed, in comparison to the vehicle group, indicating that KLF5 inhibition at early reperfusion reduced the inflammatory response post-I/R (FIGS. 5I-5R).

FIGS. 6A-6E demonstrate that NOX4 inhibition reduces infarct size and protects the heart upon I/R. DHE staining was performed after 2 hours of reperfusion to measure oxidative stress (FIG. 6A). A significant suppression of oxidative stress in both apocynin and GLX351322-treated groups was observed (FIG. 6B). Additionally, the effect of apocynin and GLX351322 on infarct size was investigated (FIG. 6C). These results demonstrate a significant reduction in infarct size in both the apocynin-treated group and the GLX351322-treated group (FIG. 6D, FIG. 6E), in comparison to the group treated only with the vehicle.

FIGS. 7A-7L demonstrate that GLUT inhibition can reduce infarct size and rescue cardiac tissue. Metabolomic analysis revealed a significant reduction of UDP-Glucose (FIG. 7A), Lactate (FIG. 7B), and acetate (FIG. 7C) concentration in αMHC-KLF5−/− mice, compared to the WT mice. Additionally, the relative mRNA expression of Ugp was significantly increased in the ischemia-reperfusion group compared with the sham group (FIG. 7D). However, treatment with ML-264 did not result in a change in the mRNA levels. Additionally, no effect on Ugdh levels was observed among the three groups (FIG. 7E). A significant suppression of Glut4 mRNA (FIG. 7F) and protein levels, as well as a trend towards reduced Glut1 levels (FIG. 7G) by ML-264 treatment, compared to the I/R group treated with the vehicle was observed (FIG. 7F-7G). Investigation of the GLUT1 (FIG. 7H) and GLUT4 (FIG. 7I) protein levels revealed a trend towards suppression of GLUT1 levels by ML-264 treatment, which did not reach statistical significance, while no difference was observed in GLUT4 levels. Administration of KL-11743 and STF-31, demonstrate a significant reduction in infarct size (FIG. 7J, FIG. 7K) by KL11743 treatment, but not by STF-31 treatment, indicating that GLUT-1 specific inhibition is not sufficient to rescue myocardial tissue from ischemia-reperfusion therapy, but global glucose transporter inhibition can reduce infarct size and rescue cardiac tissue (FIG. 7L).

### DETAILED DESCRIPTION

The details of one or more embodiments of the presently-disclosed subject matter are set forth in this document. Modifications to embodiments described in this document, and other embodiments, will be evident to those of ordinary skill in the art after a study of the information provided in this document.

While the following terms are believed to be well understood by one of ordinary skill in the art, definitions are set forth to facilitate explanation of the presently-disclosed subject matter.

Unless defined otherwise, all technical and scientific terms used herein have the same meaning as commonly understood by one of ordinary skill in the art to which the presently-disclosed subject matter belongs.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

An “effective amount,” as used herein, refers to an amount of a substance (e.g., a therapeutic compound and/or composition) that elicits a desired biological response. In some embodiments, an effective amount of a substance is an amount that is sufficient, when administered to a subject suffering from or susceptible to a disease, disorder, and/or condition, to treat, diagnose, prevent, and/or delay and/or alleviate one or more symptoms of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of a formulation to treat a disease, disorder, and/or condition is the amount that alleviates, ameliorates, relieves, inhibits, prevents, delays onset of; reduces severity of and/or reduces incidence of one or more symptoms or features of the disease, disorder, and/or condition. Furthermore, an effective amount may be administered via a single dose or via multiple doses within a treatment regimen. In some embodiments, individual doses or compositions are considered to contain an effective amount when they contain an amount effective as a dose in the context of a treatment regimen. Those of ordinary skill in the art will appreciate that a dose or amount may be considered to be effective if it is or has been demonstrated to show statistically significant effectiveness when administered to a population of patients; a particular result need not be achieved in a particular individual patient in order for an amount to be considered to be effective as described herein.

The term “administering,” as used herein, refers to any route of administering an effective amount of a therapeutic agent. In embodiments, the administering includes, but is not limited to, intravenous, intraperitoneal, subcutaneous, intramuscular, oral, intracerebral, intraspinal, intrathecal, subarachnoid, epidural, periocular, intraocular administration, and the like. In a specific embodiment, the therapeutic agent is administered orally or intravenously.

Krüppel-like factor 5 (KLF5) is a transcriptional factor, which has previously been associated with diabetic cardiomyopathy. The present inventors investigated the effects of KLF5 on acute myocardial infarction and its contribution to ischemic injury during reperfusion. Further, the present disclosure details the effects of KLF5 inhibition on systolic cardiac function and infarct size. The present disclosure demonstrates that cardiomyocyte (CM)-derived KLF5 is induced during the early phase of reperfusion. Further, both pharmacological inhibition of KLF5 and cardiomyocyte-specific KLF5 inhibition (using an αMHC-KLF5−/− murine model) preserved systolic cardiac function and reduced infarct size. Further, acute KLF5 inhibition at the onset of reperfusion reduced myocardial fibrotic scar formation and preserved cardiac function 4-weeks post ischemia-reperfusion injury.

The present disclosure also demonstrates that, mechanistically, KLF5 drives myocardial oxidative stress via NOX4 upregulation, promoting cell apoptosis. Moreover, acute NOX4-specific pharmacological inhibition at early reperfusion is shown herein to ameliorate cardiac dysfunction, reduce infarct size, and reduce oxidative stress in myocardial tissue, without affecting myocardial KLF5 levels.

Given the fact that KLF5 is an important mediator of different metabolic changes in diabetic cardiomyopathy, the present disclosure also demonstrates the impact of downstream elements of KLF5, namely nicotinamide adenine dinucleotide phosphate (NADPH) inhibition and glucose transporter (GLUT) inhibition in myocardial ischemia-reperfusion injury.

The terms “treat,” “treatment,” and “treating,” as used herein, refer to a method of alleviating or abrogating a disease, disorder, and/or symptoms thereof in a subject, including a mammal. In certain embodiments, the subject is a human patient.

Some embodiments of the present disclosure provide methods for treating patients suffering from ischemia and/or having experienced an ischemic event and who are either undergoing or scheduled to undergo reperfusion therapy. Exemplary types of reperfusion therapy include thrombolytic therapy, such as tissue plasminogen activator; percutaneous coronary intervention, such as angioplasty; surgical reperfusion, such as bypass graft surgeries; endovascular interventions, such as catheter and stent retrieval; peripheral artery revascularization, such as stent placement; thrombectomy; pharmacological reperfusion; and the like.

In some embodiments, methods comprise administering one or more primary therapeutic agents selected from KLF5 inhibitors, NOX4 inhibitors, and GLUT inhibitors in combination with the onset of reperfusion therapy. In the context of the claims, “in combination with” means within the same therapeutic time frame, for example at any time between 2 hours prior to onset, at onset, to 2 hours subsequent to onset of reperfusion therapy, inclusive of all time points contained therein. More than one dose is also contemplated. In some embodiments, the primary therapeutic agent is administered within 60 minutes of the onset of reperfusion therapy. In some embodiments, the primary therapeutic agent is administered within 30 minutes of the onset of reperfusion therapy. In other embodiments, the primary therapeutic agent is administered within 15 minutes of the onset of reperfusion therapy. In still other embodiments, the primary therapeutic agent is administered within 10 minutes of the onset of reperfusion therapy. In some embodiments, the primary therapeutic agent is administered within 5 minutes of the onset of reperfusion therapy.

In some embodiments, administering one or more primary therapeutic agents comprise administering a plurality of doses to the patient, such as two doses, three doses, four doses, etc. As an example, and without being bound by theory, in some embodiments, such as methods involving multiple doses of a primary therapeutic agent, a first dose may be given prior to the onset of reperfusion therapy, such as about 5 minutes, 10 minutes, 15 minutes, 30 minutes, 60 minutes, or 2 hours prior to the onset of reperfusion therapy. In some embodiments, a second dose may be given at the onset of reperfusion therapy. In other embodiments, a second dose may be given after the onset of reperfusion therapy, such as about 5 minutes, 10 minutes, 15 minutes, 30 minutes, 60 minutes, or 2 hours after the onset of reperfusion therapy. In still other embodiments, a second dose may be given prior to the onset of reperfusion therapy but after the first dose has been administered, such as 5 minutes, 10 minutes, 15 minutes, 30 minutes, or 60 minutes after the first dose has been administered. In some embodiments, the second dose is the same primary therapeutic agent. In other embodiments, the second does is a different primary therapeutic agent.

In some embodiments, the methods described herein comprise administering one or KLF5 inhibitors. As used herein, “KLF5 inhibitor,” “IKLF inhibitor,” and “BTEB2 inhibitor” refer interchangeably to an agent that reduces the expression level and/or activity of KLF5. The KLF5 inhibitor can inhibit KLF5 via any suitable mechanism, including direct inhibition, binding inhibition, allosteric modulation, coactivator and/or corepressor interference, downstream pathway inhibition, and/or siRNA and/or antisense nucleotide degradation.

Non-limiting examples of KLF5 inhibitors for use in the methods described herein include, but are not limited to, one or more of ML264, SR18662, SR15006, CID 5951923, ouabain, tamibaratone, AG17, AG879, prodrugs of any of the foregoing, derivatives of any of the foregoing, and combinations thereof. In a specific embodiment, the KLF5 inhibitor is selected from the group consisting of ML264, SR18662, SR15006, and CID 5951923. In a very specific embodiment, the KLF5 inhibitor is ML264.

ML264, also known as CHEMBL4557517 or MLS003874891, is a first-in-class selective KLF5 inhibitor, having the following chemical structure:

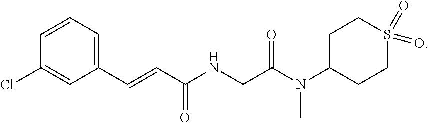 [SMILES: CN(C(=O)CNC(=O)/C=C/c1cccc(Cl)c1)C1CCS(=O)(=O)CC1]

ML264 inhibits KLF5 by interfering with the MAPK and WNT signaling pathways. Bialkowska A, et al., ML264: An Antitumor Agent that Potently and Selectively Inhibits Krüppel-like Factor Five (KLF5) Expression: A Probe for Studying Colon Cancer Development and Progression. 2011 Oct. 31 [Updated 2013 Mar. 7]. In: Probe Reports from the NIH Molecular Libraries Program [Internet]. Bethesda (MD): National Center for Biotechnology Information (US); Available from: ncbi.nlm.nih.gov/books/NBK143546/.

SR15006, CAS No.: 2505001-54-5, is an analog of ML264 that is a KLF5 inhibitor and has the structure:

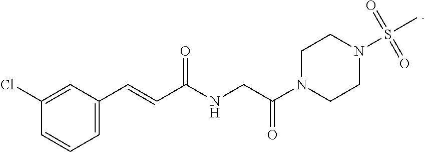 [SMILES: CS(=O)(=O)N1CCN(C(=O)CNC(=O)/C=C/c2cccc(Cl)c2)CC1]

SR15006 inhibits KLF5 by interfering with the MAPK and WNT signaling pathways. Kim J, et al., The Novel Small-Molecule SR18662 Efficiently Inhibits the Growth of Colorectal Cancer In Vitro and In Vivo. Mol Cancer Ther. 2019 November; 1 8(11):1973-1984.

SR18662, CAS No.: 2505001-62-5, is another analog of ML264 that is a potent and selective KLF5 inhibitor and has the structure:

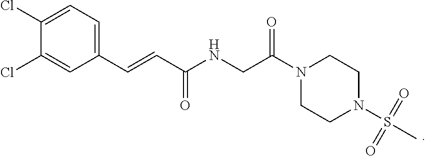 [SMILES: CS(=O)(=O)N1CCN(C(=O)CNC(=O)/C=C/c2ccc(Cl)c(Cl)c2)CC1]

SR18662 inhibits KLF5 by interfering with the MAPK and WNT signaling pathways. Kim J, et al., The Novel Small-Molecule SR18662 Efficiently Inhibits the Growth of Colorectal Cancer In Vitro and In Vivo. Mol Cancer Ther. 2019 November; 1 8(11):1973-1984.

CID 5951923, CAS No.: 749872-43-3, is a potent inhibitor of Krüppel-like factor 5 (KLF5), having the structure:

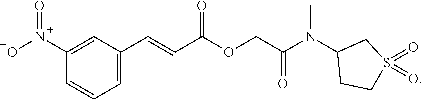 [SMILES: CN(C(=O)COC(=O)/C=C/c1cccc([N+](=O)[O-])c1)C1CCS(=O)(=O)C1]

Bialkowska A, et al., Identification of Small-Molecule Inhibitors of the Colorectal Cancer Oncogene Krüppel-like Factor 5 Expression by Ultrahigh-Throughput Screening. Mol. Cancer Ther. (2011). 10(11): 2043-2051.

Ouabain, also known as a g-strophanthin, is a cardiac glycoside having the structure:

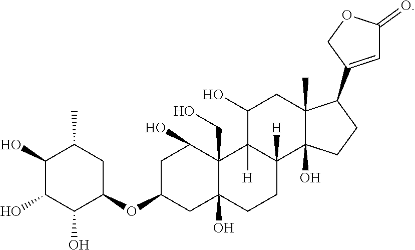

Ouabain indirectly inhibits KLF5 by inhibiting Na+/K+-ATPase, a protein pump that maintains the balance of sodium and potassium ions across cell membranes. This inhibition disrupts the normal ion gradients, leading to changes in cellular calcium levels, ion concentrations, and cell signaling pathways.

Tamibarotene, also known as Am80 or retinobenzoic acid, is a synthetic retinoid that affects KLF5 levels by targeting an active transcriptional complex containing retinoic acid receptor RARα and KLF5. Tamibarotene has the structure:

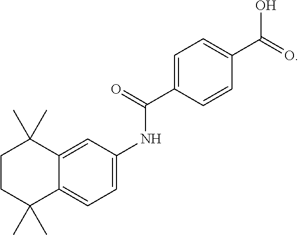 [SMILES: CC1(C)CCC(C)(C)c2cc(NC(=O)c3ccc(C(=O)O)cc3)ccc21]

AG17, also known as tyrphostin A9, is a potent platelet-derived growth factor receptor (PDGFR) inhibitor that has been shown to reduce KLF5 promoter activity and protein level in colorectal cancer cell lines. Bialkowska A, et al., Identification of Small-Molecule Inhibitors of the Colorectal Cancer Oncogene Krüppel-like Factor 5 Expression by Ultrahigh-Throughput Screening. Mol. Cancer Ther. (2011). 10(11): 2043-2051. AG17 has the structure:

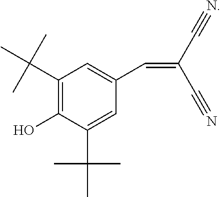 [SMILES: CC(C)(C)c1cc(C=C(C#N)C#N)cc(C(C)(C)C)c1O]

Tyrphostin AG879 is a potent inhibitor of ERRB2 tyrosine kinase that has been shown to inhibit KLF5. AG879 has the structure:

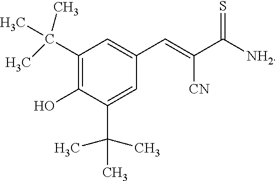 [SMILES: CC(C)(C)c1cc(/C=C(\C#N)C(N)=S)cc(C(C)(C)C)c1O]

NADPH oxidases (NOX) are membrane-bound complexes that catalyze the production of free radicals. NOX4 has been implicated in inflammation and cardiac stress. The present inventors investigated the effects of NOX and NOX4 inhibition on acute myocardial infarction and the contribution to ischemic injury during reperfusion. Further, the present disclosure details the effects of NOX and NOX4 inhibition on systolic cardiac function and infarct size.

As used herein, “NADPH oxidase 4 inhibitor,” “NOX4 inhibitor,” and “KOX inhibitor” refer interchangeably to an agent that reduces the expression level and/or activity of NOX4. The NOX4 inhibitor can inhibit NOX4 via any suitable mechanism, including direct inhibition, binding inhibition, allosteric modulation, coactivator and/or corepressor interference, downstream pathway inhibition, and/or siRNA and/or antisense nucleotide degradation.

Non-limiting examples of NOX4 inhibitors for use in the methods described herein include, but are not limited to, one or more of GLX-351322, apocynin, GKT-137831, GKT-771, diphenyleneiodonium, vas2870, vasodilator-stimulated phosphoprotein (VASP) peptide, ML-171, prodrugs of any of the foregoing, derivatives of any of the foregoing, and combinations thereof. In a specific embodiment, the NOX4 inhibitor is selected from the group consisting of GLX-351322, apocynin, and GKT-137831. In some embodiments, the NOX4 inhibitor is selected from the group consisting of GLX-351322 and apocynin. In a very specific embodiment, the NOX4 inhibitor is GLX-351322.

GLX-351322 is a selective NOX4 inhibitor, having the following chemical structure:

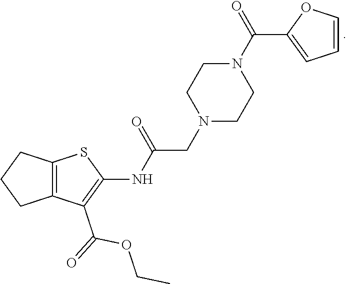 [SMILES: CCOC(=O)c1c(NC(=O)CN2CCN(C(=O)c3ccco3)CC2)sc2c1CCC2]

GLX-351322 inhibits hydrogen peroxide production from NOX4-overexpressing cells.

Apocynin, also known as acetovanillone, is a naturally occurring, non-specific NOX inhibitor having the structure:

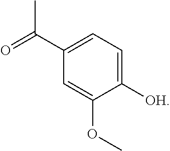

GKT-137831, also known as setanaxib and GKT831, is a potent, dual NOX1/NOX4 inhibitor, having the structure:

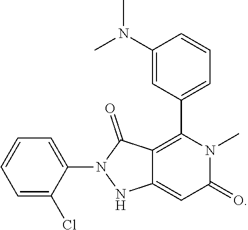 [SMILES: CN(C)c1cccc(-c2c3c(=O)n(-c4ccccc4Cl)[nH]c3cc(=O)n2C)c1]

Glucose transporters facilitate the transport of glucose across the plasma membrane. The present inventors investigated the effects of GLUT inhibition on acute myocardial infarction and the contribution to ischemic injury during reperfusion. Further, the present disclosure details the effects of GLUT inhibition on systolic cardiac function and infarct size.

As used herein, “GLUT inhibitor,” refers to an agent that reduces the expression level and/or activity of one or more glucose transporters (GLUT1-GLUT14). The GLUT inhibitor can inhibit GLUT via any suitable mechanism, including direct inhibition, binding inhibition, allosteric modulation, coactivator and/or corepressor interference, downstream pathway inhibition, and/or siRNA and/or antisense nucleotide degradation.

In some embodiments, the GLUT inhibitor is a GLUT1 inhibitor. Non-limiting examples of GLUT1 inhibitors include STF-31, WZB117, phloretin, BAY-876, and combinations thereof. In a very specific embodiment, the GLUT1 inhibitor is STF-31. In some embodiments, the GLUT inhibitor is a GLUT4 inhibitor. Non-limiting examples of GLUT4 inhibitors include wortmannin, dorsomorphin, BIRB796, and combinations thereof. In still other embodiments, the GLUT inhibitor is a non-specific GLUT inhibitor that binds all GLUT isoforms. Non-limiting examples of GLUT inhibitors include KL-11743, cyctochalasin B, cytochalasin E, and combinations thereof. In a specific embodiment, the GLUT inhibitor is KL-11743.

Data reported herein indicate that cardiomyocyte KLF5 is transiently induced upon reperfusion and contributes to myocardial ischemia-reperfusion injury. Pharmacological, as well as CM-specific KLF5 inhibition preserved systolic cardiac function and reduced infarct size. Mechanistically, KLF5 is shown herein to induce oxidative stress through NOX4 upregulation. Pharmacological inhibition of NOX enzymes reduced infarct size and rescued myocardial tissue upon the onset of reperfusion therapy.

In embodiments, the methods disclosed herein further comprise administering to the subject an effective amount of a secondary therapeutic agent. The selection of a second therapeutic agent will depend on the clinical needs of the subject. In embodiments, the primary therapeutic agent and the secondary therapeutic agent are administered consecutively or concurrently. The primary therapeutic agent(s) and the secondary therapeutic agent(s) may be administered in separate dosage forms or in the same unit dosage.

Non-limiting examples of secondary therapeutic agents include antiplatelet medications (such as aspirin, clopidogrel, ticagrelor, prasugrel, dipyridamole, ticlopidine, and eptifibatide), anticoagulant medications (such as tissue plasminogen activator (tPA), heparin, warfarin, rivaroxaban, dabigatran, apixaban, edoxaban, enoxaparin, and fondaparinux), anti-inflammatory agents, vasodilators (such as nitrates, ACE inhibitors, angiotensin receptor blockers, calcium channel blockers, sildenafil, magnesium-containing vasodilators, and the like), beta blockers, cholesterol-lowering agents, ranolazine, and combinations thereof.

### EXAMPLES

The following Examples are offered by way of illustration and are presented in a manner such that one skilled in the art should recognize are not meant to be limiting to the present disclosure as a whole or to the appended claims.

### Example 1: Materials and Methods

### Animals

All animal studies were approved by the University of Cincinnati Institutional Animal Care and Use Committee (IACUC) and were carried out in accordance with the Eighth Edition of the Guide for the Care and Use of Laboratory Animals.

Mice were housed and maintained in the University of Cincinnati vivarium, in a maximum of 5 per cage, in a specific pathogen-free facility, in a temperature-controlled environment (20-25 °C), under a 12-h light/dark cycle. Animals also received regular laboratory animal diet and water ad libitum.

No mice were excluded from the study, unless their wellness was compromised, based on the following criteria, which were used as indications for immediate euthanasia: respiratory distress/failure, aberrant inactivity with obvious discomfort, severe apparent pain, and/or recommendations from vivarium veterinarian staff.

Male and female mice 8-12 weeks old were used in order for this study to be conducted. Wild-type C57BL/6 mice were obtained from Jackson Laboratory. A cardiomyocyte-specific KLF5 knockout murine model, designated «MHC-KLF5^{-/-}, was bred in-house. αMHC-Cre mice were used as controls for these mice. αMHC-rtTA-Klf5 mice, were generated by crossing αMHC-Cre with R26-1sl-rtTA-TRE-Klf5 mice, provided by Dr. Jeffrey Whitsett. Cardiomyocyte-specific KLF5 overexpression was induced by a doxycycline-enriched diet (78 PPM/irradiated; TestDiet, St. Louis, MO).

### Pharmacological Agents

All reagents used in this study were purchased from Sigma-Aldrich (St. Louis, MO, USA), unless otherwise stated. ML264, apocynin and GLX351322 were purchased from MedChemExpress (Cat. No.: HY-19994, HY-N0088, and HY-100111, respectively).

### Surgical Procedures

Surgical procedures and interventions on the animals were performed in compliance with the guidelines “Practical guidelines for rigor and reproducibility in preclinical and clinical studies in cardioprotection,” i.e., randomization was performed for all series of experiments and the ischemia/reperfusion surgeries in mice and calculations of IS were performed by the investigators in a blinded manner.

### Echocardiography

Cardiac function was assessed by transthoracic echocardiography using the VisualSonics Vevo F2 system (VisualSonics, Toronto, ON). Sedation was induced by 3% isoflurane and maintained at 0.5% isoflurane during the procedure, during which the animals were placed on a heated table under a lamp at approximately 37 °C. Parasternal long axis (PSLAX) and short axis (SAX) images were recorded and then analyzed on a separate workstation with VevoLab software (Vevo 2100, v1.6, Visualsonic, Toronto, Canada). From the M-mode images, left ventricular cavity size for systole and diastole (LV dia;s, LV dia;d, LV Vol;s and LV Vol;d) were measured. Heart rate (HR), stroke volume (SV), and the echocardiographic calculation for fractional shortening (FS), ejection fraction (EF) and cardiac output (CO) were obtained using the Vevo software. Treatment and control groups were analyzed on the same day by the same technician in order to minimize variation. For VevoStrain analysis, B-mode images of 300 frames at greater than 200 frames/second were used.

### Murine Model of Myocardial Ischemia-Reperfusion Injury

The myocardial ischemia/reperfusion surgical protocol was applied as follows. Mice were anesthetized with 2% isoflurane inhalation until anesthesia was achieved. The animals' body temperature was maintained at 37 °C throughout the surgical procedure using a heating pad. A tracheal tube was placed via tracheal intubation and the respiration rate was set at 150 strokes/min with a tidal volume of 200 μL, using Minivent ventilator for mice (Model 845, Harvard Apparatus). A 1.2 cm incision was made over the left chest. After retraction of the pectoral major and minor muscles, the fourth intercostal space was exposed. Through a small hole at the fourth intercostal space opened with a mosquito clamp, the heart was revealed and the pericardium was removed. The left anterior descending coronary artery (LAD) was then located, sutured, and ligated at a site 3-4 mm distal to the origin of the artery under the left atrium using a 6-0 silk suture. The heart was allowed to stabilize for 15 min and right after, ischemia was induced by tightening the suture against a small piece of polyethylene tube. At the end of the 30-minute ischemia period, the ligature was released for the induction of reperfusion, and then the manual evacuation of air and closure of muscle and skin were performed. The mouse was then monitored during the recovery period, which was generally completed within 3 to 5 minutes. Sustain-released buprenorphine (BupSR, 0.5 mg/kg body weight) was provided by subcutaneous injection. Mice were then left to recover until the end of the reperfusion (2 hours, 12 hours, 24 hours, or 4 weeks). The sham group underwent the same surgical procedure except that the LAD was not occluded.

### Infarct Size Measurement

Double staining of the heart with Evans Blue dye and Triphenyl-Tetrazolium Chloride (TTC) solution was employed for infarct size measurements. Mice were anesthetized upon 3% isoflurane inhalation. The thorax was cut and opened, and hearts were gently excised. Then, the aorta was cannulated and perfused with 5 mL of normal saline. The suture was re-tightened and Evans Blue solution (2.5% in WFI) was slowly infused through the aorta, to differentiate the ischemic part of the myocardium from the non-occluded, normally perfused area. The hearts were sliced into 1-2 mm sections immediately following. The heart slices were incubated with TTC solution (1% in PBS, pH=7.4) at 37 °C for 20 min. In this way, the necrotic area is delimited as a white area, while the viable tissue is stained red. The slices were photographed using a Leica MZ16FA Stereo Microscope. The overall size of each slice (All/A), the area-at-risk (R), and the infarct area (I) were determined in a blinded manner for each slice using ImageJ software, and the percentages of Area at Risk (R/A) % and I/R % were calculated.

### Assessment of Cardiac Fibrosis

Tissue sectioning and Masson's Trichrome staining were performed. Following euthanasia under 3% isoflurane anesthesia, mouse hearts were washed with 1×PBS, fixed for 48 hours with 10% formalin, embedded in paraffin blocks, and cut into 7-μm thick sections. Following deparaffinization and rehydration, mouse heart samples were stained with Masson's trichrome staining (Sigma-Aldrich, Cat. No.: HT15-1kt). Stained heart slices were photographed using a Leica MZ16FA Stereo Microscope. Fibrotic myocardial tissue size was measured as % area with respect to the total slice area in mouse hearts imaged at 0.8×. Imaging of cardiac sections and fibrotic tissue assessment were performed by a single observer blinded to the treatment, genotype, and outcome of the animal.

### Echocardiography

Cardiac function was assessed by transthoracic echocardiography using the VisualSonics Vevo F2 system (VisualSonics, Toronto, ON). Sedation was induced by 3% isoflurane and maintained at 0.5% isoflurane during the procedure, during which the animals were placed on a heated table under a lamp at approximately 37 °C. Parasternal long axis (PSLAX) and short axis (SAX) images were recorded and then analyzed on a separate workstation with VevoLab software (Vevo 2100, v1.6, Visualsonic, Toronto, Canada). From the M-mode images, left ventricular cavity size for systole and diastole (LV dia;s, LV dia;d, LV Vol;s and LV Vol;d) were measured. Heart rate (HR), stroke volume (SV), and the echocardiographic calculation for fractional shortening (FS), ejection fraction (EF) and cardiac output (CO) were obtained using the Vevo software. Treatment and control groups were analyzed on the same day by the same technician in order to minimize variation. For VevoStrain analysis, B-mode images of 300 frames at greater than 200 frames/second were used.

### Superoxide Detection Using Dihydroethidium (DHE) Stain

Live myocardium was isolated from mice at the time of euthanasia and stained with 20 μM dihydroethidium (DHE) for 30 min at room temperature. Stained tissue was then imaged on a Zeiss LSM710 Live Duo Confocal at 490±10 nm excitation and 632±30 nm emission. Oxidized DHE fluoresces red and intercalates DNA. Individual nuclei were measured within each visual field using ImageJ software, and visual fields were averaged to measure mean fluorescence intensity for each mouse.

### Cardiomyocyte Isolation

Adult murine cardiomyocytes (ACMs) were isolated from ventricles of C57BL/6 mice. Upon induction of anesthesia with 3% isoflurane, mice were intraperitoneally injected with 0.5 mL heparin (100 IU/mL, diluted in PBS). Then, the rib cage was cut and the heart was exposed and excised. Upon heart removal, the heart was cannulated through the aorta and perfused with perfusion buffer (120.4 mM NaCl, 14.7 mM KCL, 0.6 mM Na₂HPO₄, 0.6 mM KH₂PO₄, 1.2 mM MgSO₄, 10 mM Na-HEPES, 4.6 mM NaHCO₃, 30 mM taurine, 10 mM BDM, 5.5 mM glucose; pH 7.4) for 4 min at a flow rate of 4 mL/min. Then, the perfusion buffer was switched to myocyte digestion buffer containing Collagenase II (2.4 mg/mL, Thermo Fischer Scientific, Cat. No.: 17101015) for 13 min. Ventricles were gently torn into small pieces. Perfusion buffer containing 10% calf serum and 12.5 μM CaCl₂) was added and filtered through a 100 μm nylon cell strainer, in order to receive a uniform single-cell suspension. The filtrate was then centrifuged (2 min, 20×g, 25 °C) and the supernatant, containing the non-cardiomyocyte populations, was collected and remained at 4 °C until use, while the pellet, containing the cardiomyocytes, was washed in 1×PBS and pelleted again, prior to protein and RNA isolation. Following each isolation and before proceeding to the subsequent experiments, cells were counted via hemocytometer and Trypan blue staining was performed for viability assessment and observation of cell morphology. Only cell preparations with greater than 90% rod-shaped viable cardiomyocytes were used for subsequent experiments.

### Cell-Sorting of Fibroblasts, Endothelial Cells, and White Blood Cells Using Fluorescent-Activated Cell Sorting (FACS)

The supernatant that was collected upon cardiac digestion, which contained the non-cardiomyocyte populations was centrifuged (5 min, 300×g, 4 °C) and the supernatant was discarded. At this point, 4 mL of the Red Blood Cell (RBC) lysis buffer (Thermo Fischer Scientific, Cat. No.: 00-4333-57) was added to the cell pellet and mixed. The cell suspension was then incubated for 10 minutes at room temperature, in the dark, to lyse the red blood cells and abolish their interference during the flow cytometric analysis. After red blood cell lysis, 8 mL of 2% FBS/PBS was added, and the subsequent mixture was passed through a sterile 70-μm cell strainer to obtain a uniform single-cell suspension and prepare our sample for the cytometric analysis. The cell suspension was centrifuged immediately (5 min, 300×g, 4 °C) and the supernatant was decanted. Right after, 200 μL of 2% FBS/PBS were added to the pellet and the suspension formed was transferred into a 1.5-mL Eppendorf tube to prepare cells for FACS analysis. To prevent non-specific binding of the fluorescent antibodies, cells were first incubated (15 min, 4 °C, light-protected) with 1 μL of anti-CD16/32 FC block (BD Biosciences, Cat. No.: 553142). Blocking was followed by cell incubation (30 min, 4 °C, light-protected) with the three fluorescent antibodies (2 μL of CD45 antibody, 4 μL of CD31 antibody and 10 μL of SK4 antibody). Afterward, cell suspension was centrifuged (300×g, 5 min, 4 °C), to wash off the unbounded antibody, and the supernatant was discarded. Finally, the cell pellet was resuspended in 2 mL 2% FBS/PBS. 0.5 μL of Sytox Blue viability stain was added and the suspension formed was transferred into a 5-mL sterile and RNAase-free polypropylene tube (Falcon Round-Bottom Polypropylene Tubes, STEMCELL technologies, Catalog #100-0089).

The FACSAria Fusion Flow Cytometer (BD Biosciences, San Diego, CA, USA) and the BD FACSDiva software (BD, San Diego, CA, USA) were employed to detect and sort white blood cells, fibroblasts and endothelial cells from the cell suspension. The samples were sorted into sterile and RNAase-free polypropylene tubes, each one containing 1 mL of the RLT Lysis buffer (provided within the Qiagen RNeasy plus micro kit), which were inserted into the cell collection chamber. The choice to sort cells immediately into the RLT-Buffer protects RNA from degradation, as the RTL-Buffer contains different RNA-ase inhibitors, increasing RNA integrity and quality. After sorting the whole sample, the three tubes, containing the pure-cell populations, were vortexed vigorously and the cell lysate formed was subsequently used for RNA purification and isolation, according to the manufacturer's protocol.

### Protein Purification and Western Blot Analysis

Isolated myocardial tissue/isolated primary murine cardiomyocytes were homogenized in radioimmunoprecipitation assay buffer (RIPA buffer) containing protease and phosphatase inhibitors (Thermo Fischer Scientific, Cat. No.: A32959). Total protein extracts (20-30 μg protein) were separated using SDS-polyacrylamide gel electrophoresis 8-15% and transferred onto a polyvinylidene difluoride membrane (PVDF). Membranes were then stained with Ponceau S stain (VWR, Cat. No.: K793), to determine the protein transfer efficiency. After blocking with 5% BSA, membranes were incubated overnight at 4 °C with the primary antibodies. Antibody dilutions and suppliers are displayed in Table 1. Membranes were then incubated with secondary antibodies (LI-COR Biosciences, Lincoln, NE, USA) for 1 hour at room temperature and developed using the Odyssey DLx Imaging System (LI-COR Biosciences, Lincoln, NE, USA). Relative densitometry was determined, and the β-actin protein was used as a loading control.

TABLE 1

| | | | Catalog | | |
|---|---|---|---|---|---|
| Application | Antibodies | Company | Number | Host Species | Dilution |
| Western | Anti-KLF5 | ActiveMotif | 61099 | Rabbit pAb | 1:1000 |
| Blot | Anti-NOX4 | Abcam | ab109225 | Rabbit pAb | 1:1000 |
| | Anti-BCLXL | Cell Signaling | 2764S | Rabbit mAb | 1:1000 |
| | Anti-BAX | Cell Signaling | 2772S | Rabbit mAb | 1:1000 |
| | Anti-β-ACTIN | Santa Cruz | SC-47778 | Mouse mAb | 1:2000 |
| | Anti-Mouse IgG | LI-COR | 926-32210 | Goat | 1:5000 |
| | IRDye 800CW | | | | |
| | Secondary | | | | |
| | Anti-Rabbit IgG | LI-COR | 926-32211 | Goat | 1:5000 |
| | IRDye | | | | |
| | 800CW Secondary | | | | |
| Flow | CD45 APC-Cy7 | BioLegend | 103115 | Rat mAb | 1:100 |
| Cytometry | CD31 BV605 | BioLegend | 102427 | Rat mAb | 1:50 |
| | SK4 APC | Miltenyi | 130-120-802 | Rat mAb | 1:20 |
| | | Biotec | | | |
| | CD16/CD32 Fc Block | BD | 553142 | Rat mAb | 1:200 |
| | | Biosciences | | | |

### RNA Purification and Gene Expression Analysis

Total RNA was purified from heart tissue using the TRIzol reagent according to the instructions of the manufacturer (ThermoFischer Scientific, Cat. No.: 15596018). cDNA synthesis was performed using the Applied Biosystems High Capacity cDNA Reverse Transcription Kit (ThermoFischer Scientific, Cat. No.: 4368814) from 500 ng RNA, which had been treated with DNAase I (ThermoFischer Scientific, Cat. No.: 18068015). Quantitative real-time PCR was performed with the SYBR Select Master Mix (ThermoFischer Scientific, Cat. No.: 4472903) using the manufacturer's recommendations. Incorporation of the SYBR green dye into the PCR products was monitored with the Applied Biosystems StepOnePlus Real-Time PCR System. Samples were normalized against mouse β-actin or 36B4. The sequences of the primers used for real-time PCR are listed in Table 2.

TABLE 2

| | | | Primer Sequence | SEQ ID |
|---|---|---|---|---|
| Species | Gene | Primer | (5′-3′) | NO: |
| Mus | Krüppel like factor 5 | Klf5 F | ATGCCCAGTTCGACAAACCA | 1 |
| musculus | | Klf5 R | TTGACGTCTGTGGAACAGCA | 2 |
| | Peroxisome proliferator | Ppara F | TCAAACTTGGACTTGAACG | 3 |
| | activated receptor alpha | Ppara R | GATCAGCATCCCGTCTTTGT | 4 |
| | NADPH oxidase 4 | Nox4 F | TGGCCAACGAAGGGGTTAAA | 5 |
| | | Nox4 R | CCCTTGGTTGTACAGCAGAGA | 6 |
| | Collagen type I alpha 1 | Col1a1 F | GCTTCACCTACAGCAACCCTT | 7 |
| | | Col1a1 R | TTCGATGACTGTCTTGCCCC | 8 |
| | Collagen type III alpha | Col3a1 F | CTATTTAAGGCCAGAGCAGA | 9 |
| | 1 | Col3a1 R | AAGAAGGGTGAGAAGAAACC | 10 |
| | Actin, beta | Actb F | GCAGCTCCTTCGTTGCCGGT | 11 |
| | | Actb R | ACATGCCGGAGCCGTTGTCG | 12 |
| | Actin alpha 2, smooth | Acta2 F | TGACGCTGAAGTATCCGATAGA | 13 |
| | muscle | Acta2 R | CGAAGCTCGTTATAGAAAGAGTGG | 14 |
| | Vimentin | Vim F | CGTCCACACGCACCTACAG | 15 |
| | | Vim R | GGGGGATGAGGAATAGAGGCT | 16 |
| | Protein tyrosine | Ptprc F | ATATCGCGGTGTAAAACTCGTC | 17 |
| | phosphatase receptor | Ptprc R | TAGGCTTAGGCGTTTCTGGAA | 18 |
| | type C | | | |

### Statistical Analysis

Statistical comparisons were generated using GraphPad Prism7 software, and graphs are shown with mean+/−standard error. Analyses were performed using student t-tests, or one-way ANOVA with Tukey HSD. Specific statistical tests used are stated within the figure legends. Statistical significance was identified as a p-value less than 0.05 unless otherwise specified in the figure legend.

### Example 2: Cardiomyocyte KLF5 is Transiently Induced Early in Cardiac Reperfusion Upon Myocardial I/R Injury

To explore whether KLF5 expression was induced upon myocardial I/R injury, wild-type, C57BL/6 mice (8-12 weeks old; Jackson Labs) were subjected to 30 minutes of myocardial ischemia, followed by either no reperfusion or myocardial reperfusion therapy for 2 hours, 12 hours or 24 hours.

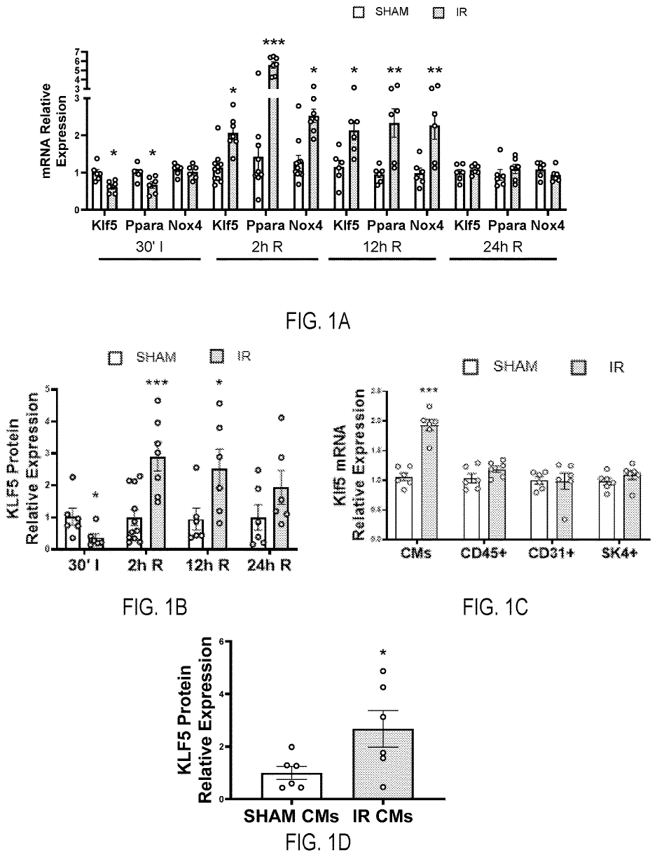

Myocardial tissue was collected at these 4 time points. Then, KLF5 and its previously established downstream elements (Ppara, Nox4) were investigated at each of these time points, as shown in FIGS. 1A-1B. Initially, cardiac KLF5 mRNA and protein levels were significantly reduced at 30 minutes of ischemia, as shown in FIGS. 1A-1B. Additionally, FIG. 1A demonstrates that Ppara mRNA levels were also downregulated at 30 minutes of ischemia, while Nox4 mRNA levels remained unaffected. However, after the onset of reperfusion, KLF5 mRNA and protein levels were increased at both the 2-hour and 12-hour time points. This increase was also accompanied by an increase in the mRNA expression of Ppara and Nox4. This demonstrates that, not only is KLF5 upregulated during this time period, but KLF5 is also transcriptionally active. Surprisingly, these markers were not differentiated at the 24-hour time point, demonstrating that KLF5 is transiently upregulated and activated during early reperfusion (FIG. 1A).

To determine which cell type was responsible for KLF5-mediated cardiotoxicity, the surgical model of 30 minutes of ischemia, followed by 2 hours of reperfusion therapy was repeated. The mice were sacrificed. Cardiomyocytes, leukocytes (CD45+), endothelial cells (CD45⁻CD31⁺), and fibroblasts (CD45⁻CD31⁻SK4⁺) were isolated from the murine left ventricular myocardial tissue. The purity of the cell populations was confirmed via cell-specific biomarker expression (data not shown).

Investigation of the KLF5 mRNA levels on the RNA isolated from each cell population, revealed a significant increase in KLF5 mRNA levels only in cardiomyocytes derived from the I/R mice, as demonstrated by FIG. 1C. This result was further confirmed at a protein level, using protein lysate isolated from pure cardiomyocytes (FIG. 1D).

### Example 3: Cardiomyocyte-Specific KLF5 Genetic Deletion Reduces Infarct Size and Protects the Heart Upon I/R Injury

KLF5 was inhibited genetically using cardiomyocyte-specific KLF5 knockout mice (αMHC-KLF5−/−). Spatial cardiac function using speckle-tracking echocardiography was evaluated, along with infarct size, upon 30 minutes of ischemia followed by 24 hours of reperfusion therapy. Oxidative stress was also determined via dihydroethidium staining.

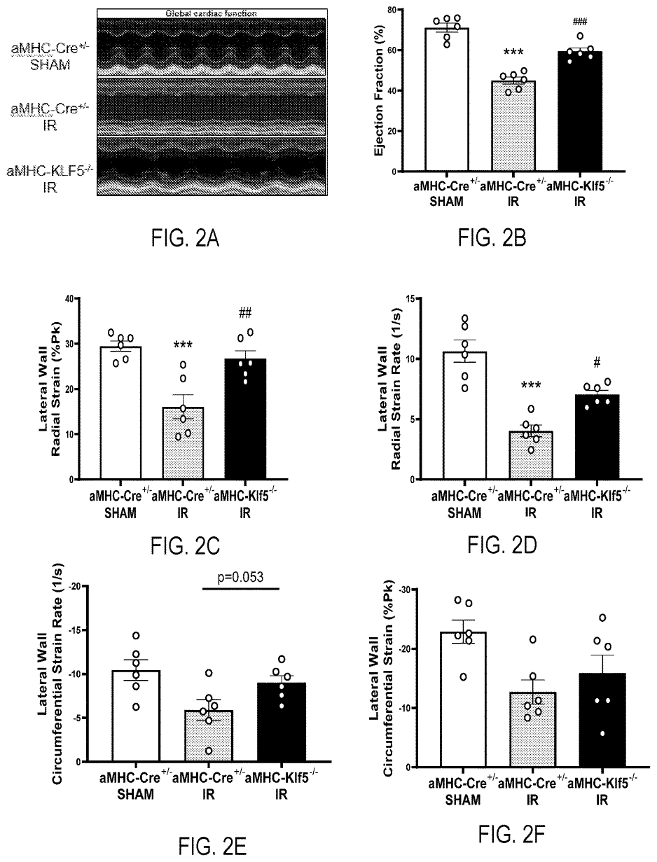

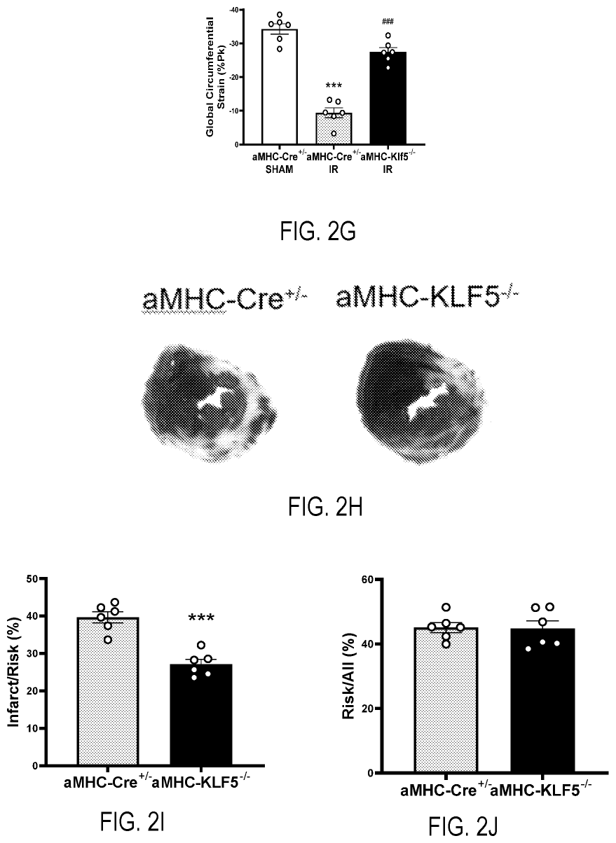

Following the observation regarding the upregulation of cardiomyocyte-derived KLF5 at early reperfusion upon myocardial IR injury, the effect of cardiomyocyte-specific genetic deletion of KLF5 was explored. The αMHC-KLF5^{-/-} were subjected to 30 minutes of myocardial ischemia and 24 hours of reperfusion therapy, for the concurrent evaluation of cardiac function (FIG. 2A) and infarct size, compared with WT (αMHC-Cre+/−). Cardiac 2D echocardiography analysis showed a higher ejection fraction in the αMHC-KLF5^{-/-} mice subjected to reperfusion therapy compared to the WT mice undergoing reperfusion (FIG. 2B). A more comprehensive evaluation of cardiac function was performed, using speckle tracking echocardiography, which revealed a significant increase in lateral wall radial and circumferential strain and strain rate in the αMHC-KLF5^{-/-} mice subjected to reperfusion therapy compared to the WT mice undergoing reperfusion (FIGS. 2C-2G). Consistently, infarct size evaluation upon Evan's Blue/TTC staining revealed a significant reduction of infarct size in the αMHC-KLF5^{-/-} group, compared to the WT groups (FIG. 2H-2J).

### Example 4: Pharmacological Inhibition of KLF5 Protects the Heart Against Myocardial I/R Injury

After determining that genetic deletion of KLF5 protected the heart, pharmacological inhibition of KLF5 was evaluated. Spatial cardiac function using speckle-tracking echocardiography was evaluated, along with infarct size, upon 30 minutes of ischemia followed by 24 hours of reperfusion therapy. Oxidative stress was also determined via dihydroethidium staining.

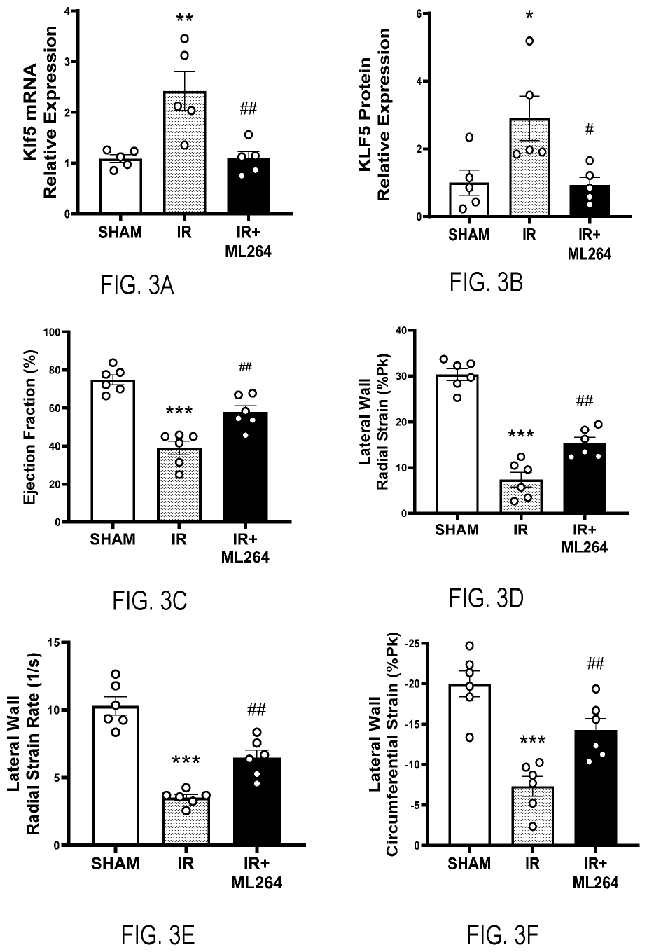

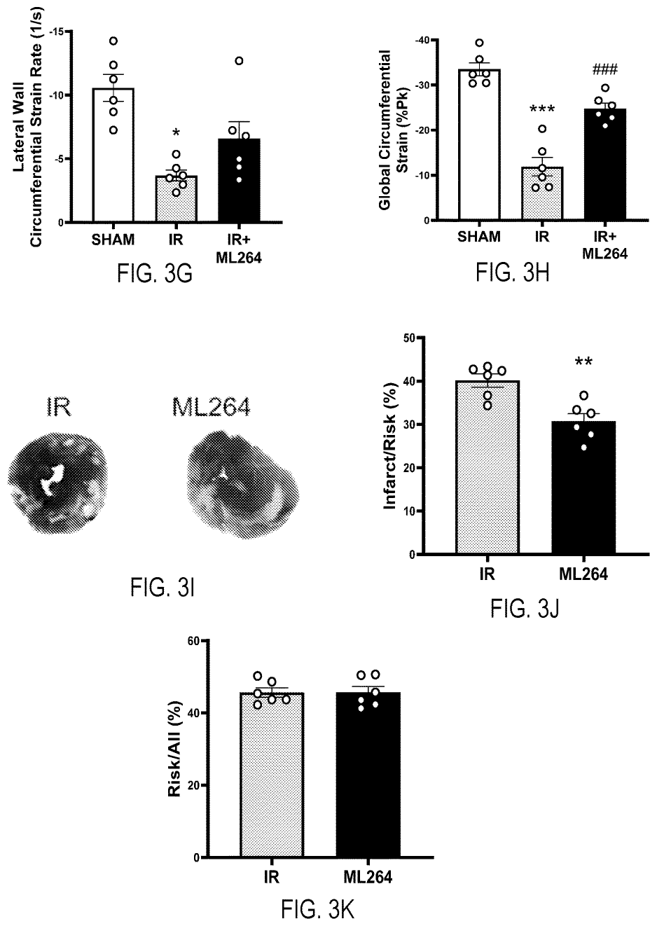

To explore whether KLF5 inhibition holds therapeutic potential against myocardial I/R injury, WT mice were subjected to 30 minutes of ischemia, followed by 24 hours of reperfusion while being treated with a pharmacological KLF5 inhibitor, namely ML264. ML264 (25 mg/kg/12 h) was dissolved in 10% DMSO, 10% Tween 80, and 80% normal saline, and was administered by intraperitoneal injection in mice. KLF5 suppression was confirmed in the cardiac tissue at 30 minutes of ischemia, as shown in FIGS. 3A-3B.

A first dose of ML264 was administered five minutes before the reopening of the coronary artery, due to the clinical application that this time point holds, as well as previous reports about the importance of this time point regarding the pharmacological possibilities for the cardioprotection against I/R injury. 2D echocardiography was performed after 24 hours of reperfusion. A significant preservation of ejection fraction was observed in I/R mice treated with ML264, compared to those treated with the vehicle (FIG. 3C). Consistently, speckle-tracking echocardiography revealed a significant increase in lateral wall radial and circumferential strain and strain rate (FIG. 3D-3H). Evaluation of infarct size was also performed and revealed a significant reduction of infarct size in the ML264-treated mice, in comparison to the ones treated with the vehicle (FIG. 3I-3K).

### Example 5: Acute Pharmacological as Well as Chronic KLF5 Inhibition Attenuate Cardiac Dysfunction and Fibrosis in a Chronic Model of I/R Injury

Since myocardial ischemia-reperfusion injury-induced cell death activates collagen deposition in the cardiac muscle, which promotes cardiac dysfunction and eventually leads to the development of heart failure, the present disclosure further investigates whether the decrease in the infarct size observed by KLF5 inhibition can lead to diminished collagen deposition in the heart muscle as well as a preservation of the cardiac function.

To study this question, C57BL/6 mice were subjected to 30 minutes of ischemia, followed by 4 weeks of reperfusion therapy. Two doses of ML264 were administered to inhibit KLF5 expression at early reperfusion.

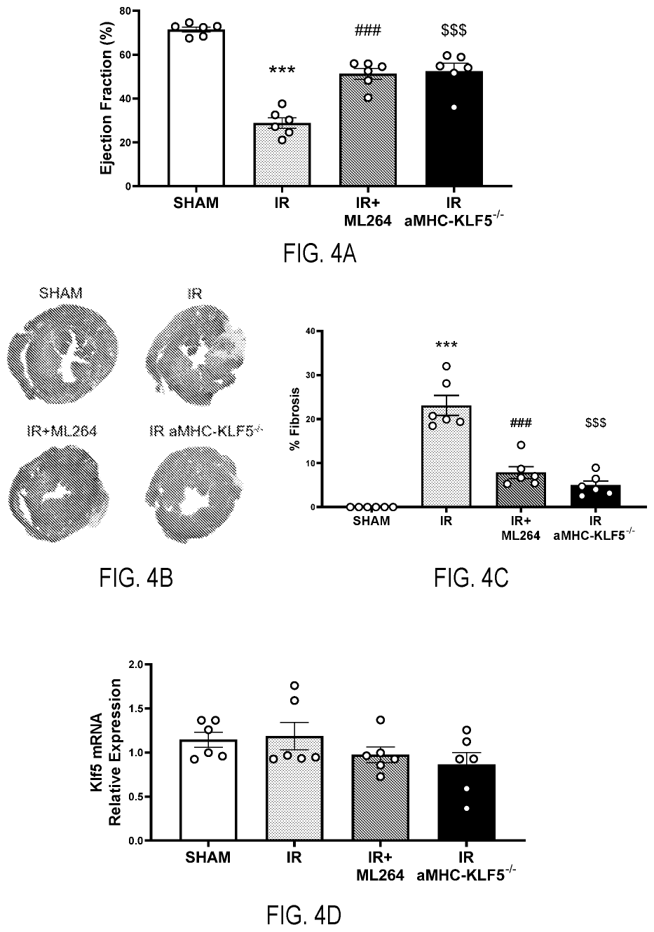

Further, to evaluate for long-term outcome between acute and long-term KLF5 inhibition, αMHC-KLF5^{-/-} mice were included. 2D echocardiography revealed a significant preservation in ejection fraction in the ML264 and the αMHC-KLF5^{-/-} groups at 4 weeks post-I/R, in comparison to the IR group treated with the vehicle (FIG. 4A). Masson's Trichrome staining was performed to the heart sections for the investigation of cardiac fibrosis and revealed a significant reduction of fibrotic scar tissue formation both in the ML264 and the αMHC-KLF5^{-/-} groups (FIG. 4B-4C).

Consistently, mRNA levels of Col1a1 and Col3a1 were also decreased in these two groups, in comparison to the IR group treated with the vehicle (data not shown). Importantly, KLF5 mRNA levels were unchanged among the four groups at 4 weeks post-ischemia-reperfusion, indicating that KLF5 inhibition at early reperfusion is sufficient to ameliorate the cardiotoxic effect of KLF5 in myocardial ischemia-reperfusion injury (FIG. 4D).

### Example 6: KLF5 Promotes Oxidative Stress and Leads to Apoptosis Through NOX4-Mediated Mechanisms

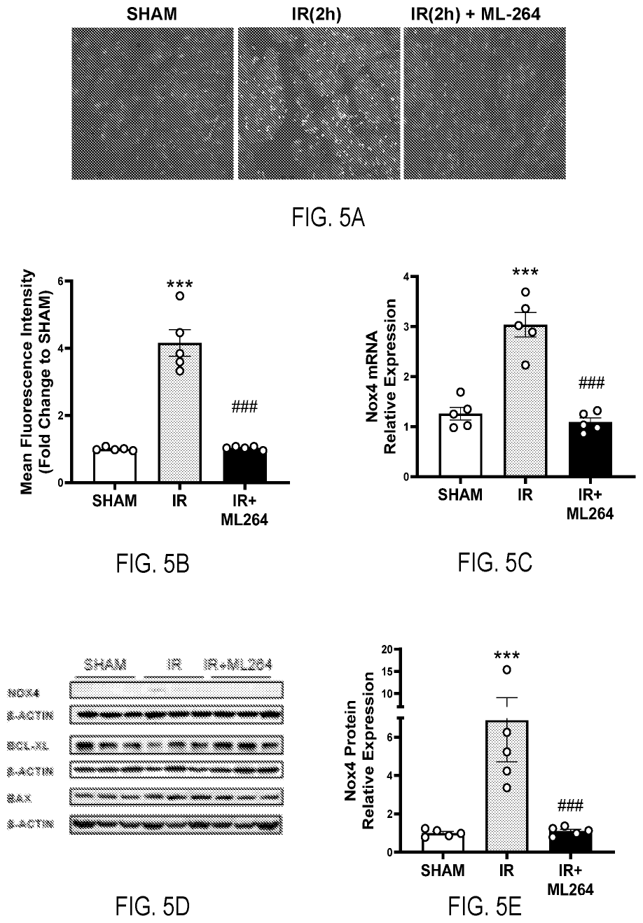

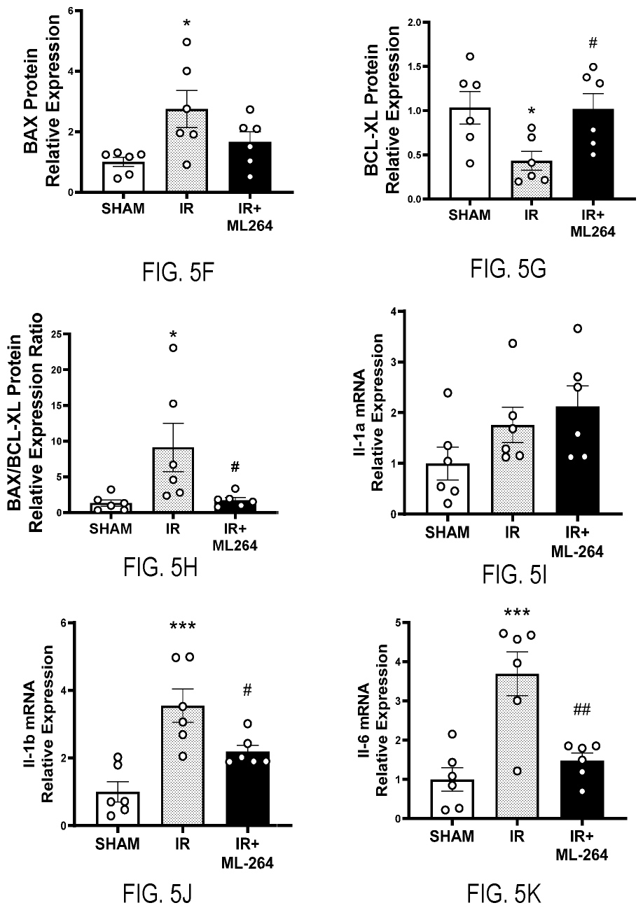

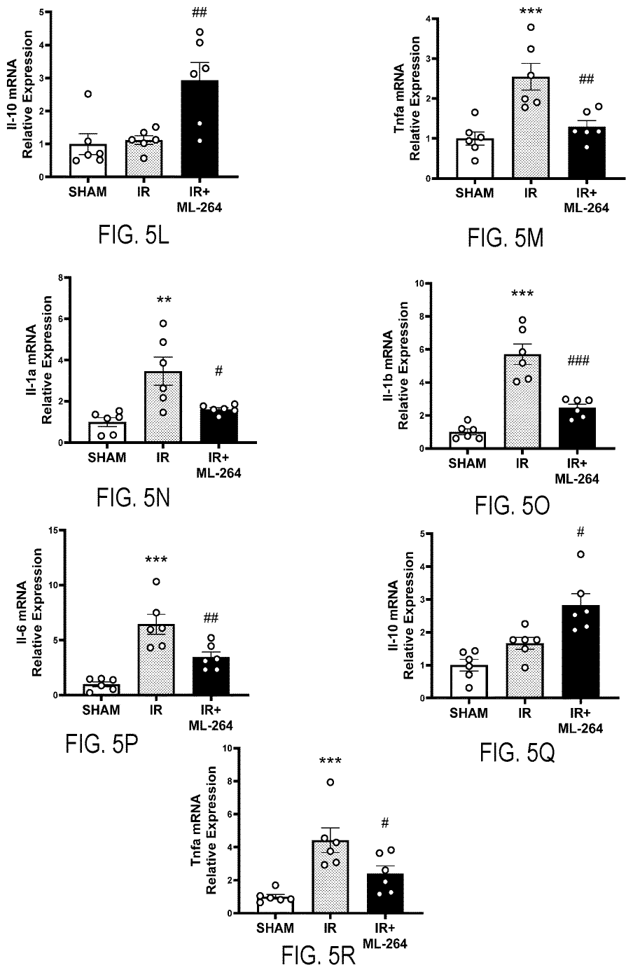

In order to unravel the mechanisms of KLF5-mediated cardiotoxicity, oxidative stress levels were evaluated at early reperfusion. To do so, C57BL/6 mice were subjected to 30 minutes of ischemia followed by 2 hours of reperfusion. ML264 was administered 5 minutes prior to the reopening of the coronary artery. DHE staining revealed a significant reduction in reactive oxygen species formation in the ML264-treated group, in comparison to the ischemia-reperfusion group treated with the vehicle (FIG. 5A-5B). Concurrently, a significant suppression of NOX4 levels upon ML264 treatment was observed, indicating that ML264 administration reduces oxidative stress through NOX4 downregulation (FIG. 5C-5E). Investigation of the apoptotic pathway revealed a suppression of the BAX apoptotic proteins (FIG. 5F) and an upregulation of the BCL-XL anti-apoptotic proteins (FIG. 5G) in the ML264 treated group, indicating that KLF5 inhibition at early reperfusion ameliorates ischemia-induced apoptosis (FIGS. 5F-5H). Given the fact that inflammation and diapedesis of immune cells are important hallmarks of the post-ischemia-reperfusion response, the mRNA levels of IL-1α, IL-1β, IL-6, IL-10, and TNFα in the ischemic myocardium upon ML264 treatment were evaluated at 2 hours of reperfusion (FIGS. 51-5M) and 24 hours of reperfusion (FIGS. 5N-5Q). Significant suppression of IL-1B, IL-6, and TNFα levels in the ML264 group was observed, in comparison to the vehicle group, indicating that KLF5 inhibition at early reperfusion reduced the inflammatory response post-I/R (FIGS. 5I-5Q).

### Example 7: NOX4 Inhibition Reduces IS and Protects the Heart Upon I/R

NADPH oxidase inhibitors were also administered to evaluate the contribution of NADPH oxidase enzymes in myocardial ischemia-reperfusion injury. To demonstrate that KLF5 inhibition reduces ischemia-reperfusion injuries through NOX4 suppression, the previously employed NADPH oxidase inhibitors were administered to C57BL/6 mice in the setting of I/R injury. Specifically, a non-selective NADPH oxidase inhibitor, namely apocynin, as well as an NADPH oxidase 4 selective inhibitor, namely GLX351322 were used.

Apocynin (5 mg/kg/12 h) and GLX351322 (3.8 mg/kg/12 h) were dissolved in 10% DMSO and 90% normal saline and administered by intraperitoneal injection in mice. The control mice for each one of the treatment groups received the corresponding volume of each vehicle, respectively.

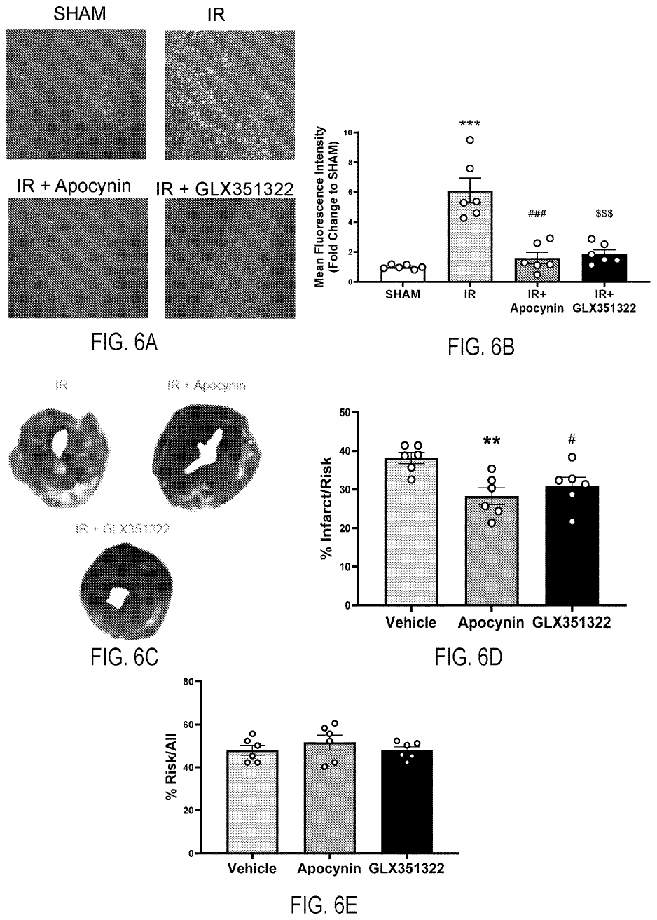

The compounds were administered at early reperfusion, as done with the administration of ML264. DHE staining was performed after 2 hours of reperfusion to measure oxidative stress (FIG. 6A). A significant suppression of oxidative stress in both apocynin and GLX351322-treated groups was observed (FIG. 6B).

Additionally, the effect of apocynin and GLX351322 on infarct size was investigated. These results demonstrate a significant reduction in infarct size in both the apocynin-treated group and the GLX351322-treated group, in comparison to the group treated only with the vehicle group (FIG. 6C-6E).

### Example 8: Metabolomics and Glucose Regulation

Both WT and αMHC-KLF5−/− mice were subjected to 30 minutes ischemia followed by 2 hours of perfusion therapy. Myocardial tissue was harvested to perform untargeted metabolomics.

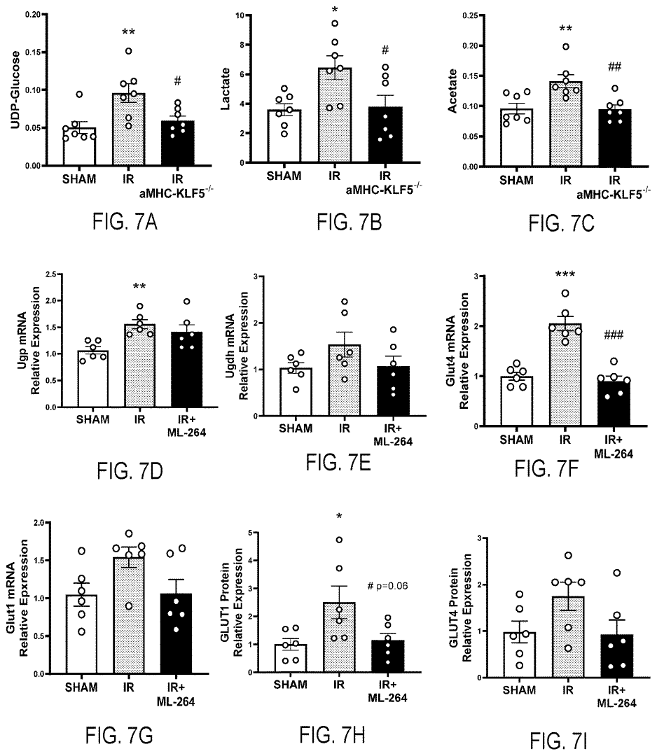

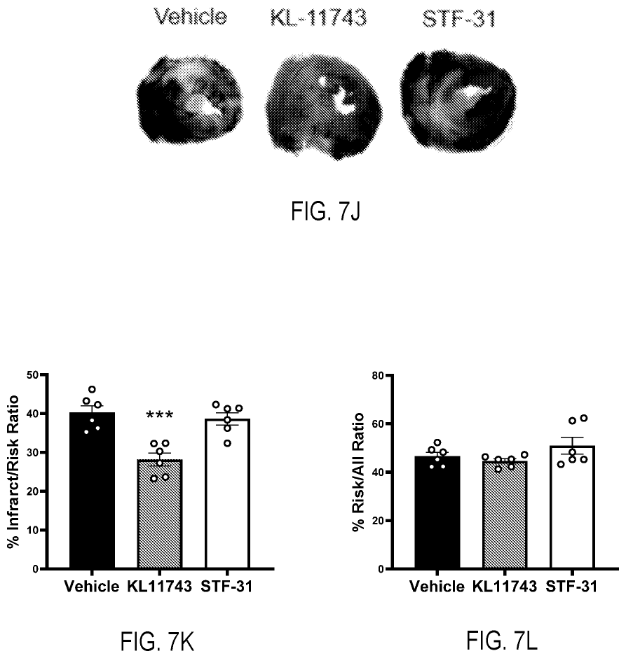

Metabolomic analysis revealed a significant reduction of UDP-Glucose, Lactate, and Acetate concentration in αMHC-KLF5−/−, compared to the WT mice (FIG. 7A-7C). Additionally, the relative mRNA expression of Ugp was significantly increased in the ischemia-reperfusion group compared with the sham group (FIG. 7D). However, treatment with ML-264 did not result in a change in the mRNA levels. Additionally, no effect on Ugdh levels was observed among the three groups (FIG. 7E).

Because glucose is the precursor molecule for the synthesis of UDP-Glucose, the expression levels of Glut1 and Glut4 transporters were analyzed. A significant suppression of Glut4 mRNA levels, as well as a trend towards reduced Glut1 levels by ML-264 treatment, compared to the I/R group treated with the vehicle was observed (FIG. 7F-7G).

Investigation of the GLUT1 and GLUT4 protein levels revealed a trend towards suppression of GLUT1 levels by ML-264 treatment, which did not reach statistical significance, while no difference was observed in GLUT4 levels (FIG. 7H-7I). Finally, to test the potential involvement of GLUT1 and GLUT4 in myocardial ischemia-reperfusion injury, two compounds, namely KL-11743, a non-specific GLUT inhibitor, as well as STF-31, which is a GLUT1-specific inhibitor, were evaluated.

Administration of these two compounds was performed 5 minutes before the reopening of the coronary artery and infarct size was assessed upon 30 minutes of ischemia followed by 24 hours of reperfusion therapy. These results indicated a significant reduction in infarct size by KL11743 treatment, but not by STF-31 treatment, indicating that GLUT-1-specific inhibition is not sufficient to rescue myocardial tissue from ischemia-reperfusion therapy, but global glucose transporter inhibition can reduce infarct size and rescue cardiac tissue (FIG. 7J-7L).

### Aspect Listing

In a first aspect, alone or in combination with any other aspect herein, the present disclosure concerns a method of treating a patient in need of reperfusion therapy following an ischemic event, the method comprising administering to the patient one or more primary therapeutic agents in combination with onset of reperfusion therapy, wherein the one or more primary therapeutic agents are selected from the group consisting of Krüppel-like factor 5 (KLF5) inhibitors, NADPH oxidase (NOX) inhibitors, and glucose transport (GLUT) inhibitors.

In a second aspect, alone or in combination with any other aspect herein, the present disclosure concerns a method wherein the patient suffered a myocardial infarction.

In a third aspect, alone or in combination with any other aspect herein, the present disclosure concerns a method wherein the one or more primary therapeutic agents is a KLF5 inhibitor.

In a fourth aspect, alone or in combination with any other aspect herein, the present disclosure concerns a method wherein the KLF5 inhibitor is selected from the group consisting of ML264, SR18662, SR15006, and CID 5951923.

In a fifth aspect, alone or in combination with any other aspect herein, the present disclosure concerns a method wherein the KLF5 inhibitor is ML264.

In a sixth aspect, alone or in combination with any other aspect herein, the present disclosure concerns a method wherein the one or more primary therapeutic agents is a NOX inhibitor.

In a seventh aspect, alone or in combination with any other aspect herein, the present disclosure concerns a method wherein the NOX inhibitor is apocynin.

In an eighth aspect, alone or in combination with any other aspect herein, the present disclosure concerns a method wherein the NOX inhibitor is a NOX4 inhibitor.

In a ninth aspect, alone or in combination with any other aspect herein, the present disclosure concerns a method wherein the NOX4 inhibitor is selected from the group consisting of GLX-351322, apocynin, and GKT-137831.

In a tenth aspect, alone or in combination with any other aspect herein, the present disclosure concerns a method wherein the NOX4 inhibitor is GLX-351322.

In an eleventh aspect, alone or in combination with any other aspect herein, the present disclosure concerns a method wherein the one or more primary therapeutic agents is a GLUT inhibitor.

In a twelfth aspect, alone or in combination with any other aspect herein, the present disclosure concerns a method wherein the GLUT inhibitor is selected from the group consisting of KL-11743, cyctochalasin B, and cytochalasin E.

In a thirteenth aspect, alone or in combination with any other aspect herein, the present disclosure concerns a method wherein the GLUT inhibitor is KL-11743.

In a fourteenth aspect, alone or in combination with any other aspect herein, the present disclosure concerns a method wherein administering the one or more primary therapeutic agents in combination with the onset of reperfusion therapy comprises administering a plurality of primary therapeutic agents.

In a fifteenth, alone or in combination with any other aspect herein, the present disclosure concerns a method comprising administering the one or more primary therapeutic agents systemically via an enteral or parenteral route.

In a sixteenth aspect, alone or in combination with any other aspect herein, the present disclosure concerns a method, wherein the one or more primary therapeutic agents is administered as a single dose or in multiple doses.

In a seventeenth aspect, alone or in combination with any other aspect herein, the present disclosure concerns a method wherein the one or more primary therapeutic agents is administered in multiple doses, wherein a first dose of the multiple doses is administered before the onset of reperfusion therapy.

In an eighteenth aspect, alone or in combination with any other aspect herein, the present disclosure concerns a method wherein a second dose of the multiple doses is administered after the onset of reperfusion therapy.

In a nineteenth aspect, alone or in combination with any other aspect herein, the present disclosure concerns a method of treating a patient experiencing a myocardial ischemic event, the method comprising administering to the subject an effective amount of one or more primary therapeutic agents selected from the group consisting of ML264, apocynin, KL-11743, and GLX-351322, prior to administering reperfusion therapy to the patient.

In a twentieth aspect, alone or in combination with any other aspect herein, the present disclosure concerns a method further comprising administering a second dose of the one or more primary therapeutic agents after onset of reperfusion therapy.

It is noted that the terms “substantially” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue. The term “substantially” is used herein also to represent the degree by which a quantitative representation may vary from a stated reference without resulting in a change in the basic function of the subject matter at issue. Thus, it is used to represent the inherent degree of uncertainty that may be attributed to any quantitative comparison, value, measurement, or other representation, referring to an arrangement of elements or features that, while in theory would be expected to exhibit exact correspondence or behavior, may in practice embody something less than exact.

Unless otherwise defined, all technical and scientific terms used herein have the same meaning as commonly understood by one of ordinary skill in the art to which the invention belongs. The terminology used in the description herein is for describing particular embodiments only and is not intended to be limiting. As used in the specification and appended claims, the singular forms “a,” “an,” and “the” are intended to include the plural forms as well, unless the context clearly indicates otherwise.

It is noted that one or more of the following claims utilize the term “wherein” as a transitional phrase. For the purposes of defining the present technology, it is noted that this term is introduced in the claims as an open-ended transitional phrase that is used to introduce a recitation of a series of characteristics of the structure and should be interpreted in like manner as the more commonly used open-ended preamble term “comprising.”

It should be understood that where a first component is described as “comprising” or “including” a second component, it is contemplated that, in some embodiments, the first component “consists” or “consists essentially of” the second component. Additionally, the term “consisting essentially of” is used in this disclosure to refer to quantitative values that do not materially affect the basic and novel characteristic(s) of the disclosure.

It should be understood that any two quantitative values assigned to a property or measurement may constitute a range of that property or measurement, and all combinations of ranges formed from all stated quantitative values of a given property or measurement are contemplated in this disclosure.

While particular embodiments have been illustrated and described herein, it should be understood that various other changes and modifications may be made without departing from scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

1. A method of treating a patient in need of reperfusion therapy following an ischemic event, the method comprising administering to the patient one or more primary therapeutic agents in combination with onset of reperfusion therapy, wherein the one or more primary therapeutic agents are selected from the group consisting of Krüppel-like factor 5 (KLF5) inhibitors, NADPH oxidase (NOX) inhibitors, and glucose transport (GLUT) inhibitors.

2. The method according to claim 1, wherein the patient suffered a myocardial infarction.

3. The method according to claim 1, wherein the one or more primary therapeutic agents is a KLF5 inhibitor.

4. The method according to claim 3, wherein the KLF5 inhibitor is selected from the group consisting of ML264, SR18662, SR15006, and CID 5951923.

5. The method according to claim 4, wherein the KLF5 inhibitor is ML264.

6. The method according to claim 1, wherein the one or more primary therapeutic agents is a NOX inhibitor.

7. The method according to claim 6, wherein the NOX inhibitor is apocynin.

8. The method according to claim 6, wherein the NOX inhibitor is a NOX4 inhibitor.

9. The method according to claim 8, wherein the NOX4 inhibitor is selected from the group consisting of GLX-351322, apocynin, and GKT-137831.

10. The method according to claim 8, wherein the NOX4 inhibitor is GLX-351322.

11. The method according to claim 1, wherein the one or more primary therapeutic agents is a GLUT inhibitor.

12. The method according to claim 11, wherein the GLUT inhibitor is selected from the group consisting of KL-11743, cyctochalasin B, and cytochalasin E.

13. The method according to claim 11, wherein the GLUT inhibitor is KL-11743.

14. The method according to claim 1, wherein administering the one or more primary therapeutic agents in combination with the onset of reperfusion therapy comprises administering a plurality of primary therapeutic agents.

15. The method according to claim 1, comprising administering the one or more primary therapeutic agents systemically via an enteral or parenteral route.

16. The method according to claim 1, wherein the one or more primary therapeutic agents is administered as a single dose or in multiple doses.

17. The method according to claim 16, wherein the one or more primary therapeutic agents is administered in multiple doses, wherein a first dose of the multiple doses is administered before the onset of reperfusion therapy.

18. The method according to claim 17, wherein a second dose of the multiple doses is administered after the onset of reperfusion therapy.

19. A method of treating a patient experiencing a myocardial ischemic event, the method comprising administering to the subject an effective amount of one or more primary therapeutic agents selected from the group consisting of ML264, apocynin, KL-11743, and GLX-351322, prior to administering reperfusion therapy to the patient.

20. The method according to claim 18, further comprising administering a second dose of the one or more primary therapeutic agents after onset of reperfusion therapy.


SEQUENCE LISTING (18 sequences)

SEQ ID NO: 1 - DNA, 20 bp, Mus musculus
  source 1..20: mol_type = genomic DNA
atgcccagttcgacaaacca

SEQ ID NO: 2 - DNA, 20 bp, Mus musculus
  source 1..20: mol_type = genomic DNA
ttgacgtctgtggaacagca

SEQ ID NO: 3 - DNA, 19 bp, Mus musculus
  source 1..19: mol_type = genomic DNA
tcaaacttggacttgaacg

SEQ ID NO: 4 - DNA, 20 bp, Mus musculus
  source 1..20: mol_type = genomic DNA
gatcagcatcccgtctttgt

SEQ ID NO: 5 - DNA, 20 bp, Mus musculus
  source 1..20: mol_type = genomic DNA
gatcagcatcccgtctttgt

SEQ ID NO: 6 - DNA, 21 bp, Mus musculus
  source 1..21: mol_type = genomic DNA
cccttggttgtacagcagaga

SEQ ID NO: 7 - DNA, 20 bp, Mus musculus
  source 1..20: mol_type = genomic DNA
ttcgatgactgtcttgcccc

SEQ ID NO: 8 - DNA, 20 bp, Mus musculus
  source 1..20: mol_type = genomic DNA
ttcgatgactgtcttgcccc

SEQ ID NO: 9 - DNA, 20 bp, Mus musculus
  source 1..20: mol_type = genomic DNA
ctatttaaggccagagcaga

SEQ ID NO: 10 - DNA, 20 bp, Mus musculus
  source 1..20: mol_type = genomic DNA
aagaagggtgagaagaaacc

SEQ ID NO: 11 - DNA, 20 bp, Mus musculus
  source 1..20: mol_type = genomic DNA
gcagctccttcgttgccggt

SEQ ID NO: 12 - DNA, 20 bp, Mus musculus
  source 1..20: mol_type = genomic DNA
acatgccggagccgttgtcg

SEQ ID NO: 13 - DNA, 22 bp, Mus musculus
  source 1..22: mol_type = genomic DNA
tgacgctgaagtatccgataga

SEQ ID NO: 14 - DNA, 24 bp, Mus musculus
  source 1..24: mol_type = genomic DNA
cgaagctcgttatagaaagagtgg

SEQ ID NO: 15 - DNA, 19 bp, Mus musculus
  source 1..19: mol_type = genomic DNA
cgtccacacgcacctacag

SEQ ID NO: 16 - DNA, 21 bp, Mus musculus
  source 1..21: mol_type = genomic DNA
gggggatgaggaatagaggct

SEQ ID NO: 17 - DNA, 22 bp, Mus musculus
  source 1..22: mol_type = genomic DNA
atatcgcggtgtaaaactcgtc

SEQ ID NO: 18 - DNA, 21 bp, Mus musculus
  source 1..21: mol_type = genomic DNA
taggcttaggcgtttctggaa